(19) United States
(12) Patent Application Publication
(10) Pub. No.: US 2011/0201797 A1
(43) Pub. Date: Aug. 18, 2011

(54) Nucleic Acid Shearing Device with Disposable Cartridge

(75) Inventors: Patrick B. Cahill, Natick, MA (US); Son Hoang, Dorchester, MA (US); Filip Rege, Belmont, MA (US); Shailendra Yadav, Lexington, MA (US)

(73) Assignee: Massachusetts Institute of Technology, Cambridge, MA (US)

(21) Appl. No.: 13/030,678

(22) Filed: Feb. 18, 2011

Related U.S. Application Data
(60) Provisional application No. 61/305,577, filed on Feb. 18, 2010.

(51) Int. Cl. C07H 21/00; B01L 3/00

(52) U.S. Cl. 536/23.1; 422/554; 422/430

(57) ABSTRACT

Apparatus, devices, and methods for shearing nucleic acids are provided. In one exemplary embodiment the device is a disposable nucleic acid shearing cartridge. The cartridge includes two reservoirs, an orifice structure disposed therebetween, and a fluid driver. The reservoirs are in fluid communication with each other by way of the orifice structure, and the fluid driver cycles a sample between the two reservoirs. By passing a sample from one reservoir to the next, through the orifice structure, an orifice located in the orifice structure can shear the sample to a desired length. The sheared sample can then be used, for example, in a sequencing device. Apparatuses for cycling samples in the cartridge, and methods and kits for shearing nucleic acids, are also disclosed.

### PRIORITY OF THE INVENTION

The present invention claims priority to U.S. Provisional Application No. 61/305,577, entitled “DNA SHEARING DEVICE WITH DISPOSIBLE CARTRIDGE” and filed on Feb. 18, 2010.

### GOVERNMENT RIGHTS

This invention was made with government support awarded by the National Institutes of Health (NIH) under Grant U54 HG003067. The government has certain rights in the invention.

### FIELD

The present invention generally relates to devices and methods for shearing nucleic acids, and more particularly is directed to a nucleic acid shearing device that includes a disposable cartridge.

### BACKGROUND

The sequencing of nucleic acids, such as deoxyribonucleic acid (DNA) and ribonucleic acid (RNA), is an important and prevalent activity that produces genetic information from biological organisms. Nucleic acids like DNA and RNA are sequenced by mapping the specific nucleotide bases of a particular strand of DNA or RNA. Knowing the composition of nucleic acids that form a particular strand of DNA or RNA has a powerful impact on biological research and discovery. For example, treatments and medicines can then be targeted to treat particular diseases associated with strands of DNA and RNA in which the nucleotide base order is known.

A number of different technologies have been developed to assist in sequencing nucleic acids, including machines developed by companies like Pacific Biosciences, Illumina, and Life Technologies. However, prior to sequencing a nucleic acid, the acid must first be sheared to a size that can be handled by the sequencing machine.—regardless of the sequencing machine that is used. Different machines have different preferred strand size ranges.

Devices that are currently used to shear nucleic acids have a limited throughput. This is both in the context of the amount of time it takes to run a single sample, and also in the context of only being capable of shearing a single sample at a time. Additionally, current devices are limited in the size of sheared sample they can produce. Further, current shearing devices typically require a great deal of preparation time between samples. This preparation time includes a cleaning procedure in which the container in which the sample was disposed is washed and cleaned prior to the introduction of another sample for shearing. The risk for contamination with current devices is also higher than desired, at least in part because of the many steps required to prepare samples that are run in the same device. Still further, both because of the risk of contamination, and because current shearing techniques do not produce a consistent size of sheared samples even when conditions are kept generally the same, accuracy is also an area in which current shearing devices can be deficient.

Accordingly, it is desirable for shearing devices and methods to improve such that they produce more accurate and repeatable results. It is further desirable for the throughput of an individual sample being sheared to be improved, and to create a device that is capable of processing multiple samples at the same time. Still further, it is desirable for the shearing of the sample to be performed in an automated fashion.

### SUMMARY

Apparatuses, devices, and methods are provided for shearing nucleic acids. In one exemplary embodiment, a disposable nucleic acid shearing cartridge includes two reservoirs, an orifice structure in fluid communication with each of the two reservoirs, and a fluid driver for cycling a sample between the two reservoirs multiple times to shear the nucleic acid into pieces of desired lengths. The first reservoir can receive a nucleic acid sequence sample and the second reservoir can receive the sample from the first reservoir following passage of the sample through the orifice structure.

In one embodiment the first reservoir can be a syringe body and the fluid driver can be a syringe plunger that is coupled to the syringe body. The first reservoir can define a particular volume. For example, in one embodiment the first reservoir can define a volume of at least about 0.5 milliliters, while in another embodiment the first reservoir can define a volume of at least about 1 milliliter. The first reservoir and/or the second reservoir can include a plastic body.

The first orifice structure can include an inorganic material having at least one fluid-passing hole therethrough. In one embodiment the fluid-passing hole can have a diameter in a range of about 25 micrometers to about 125 micrometers. In another embodiment the fluid-passing hole can have a diameter in a range of about 50 micrometers to about 100 micrometers. The inorganic material of the first orifice structure can be a material selected from materials such as glasses, ceramics, and crystalline materials. In one embodiment the inorganic material of the orifice structure includes sapphire.

In one exemplary embodiment of a nucleic acid shearing apparatus, the apparatus includes a housing for receiving at least one nucleic acid shearing cartridge, a reciprocating actuator, and a processor. In one embodiment the housing can be configured to be used with at least one nucleic acid shearing cartridge that includes a first reservoir for receiving a nucleic acid sequence sample, an orifice structure in fluid communication with the first reservoir, a second reservoir also in fluid communication with the orifice structure and configured to receive the sample following passage through the orifice structure, and a fluid driver for cycling the sample between the first and second reservoirs. The reciprocating actuator can be configured to engage the fluid driver of the cartridge, thereby causing a sample within the first reservoir to pass through the orifice structure and into the second reservoir. The reciprocating actuator can also cause a sample to pass from the second reservoir, through the orifice structure, and back to the first reservoir. The processor can be configured to control a number of shearing parameters, including, for example, a flow rate of the sample and a number of times the sample passes through the orifice structure. In one embodiment the housing includes multiple receptacles. Each receptacle can receive a cartridge, thereby allowing multiple samples to be processed in parallel. Further, in one exemplary embodiment the reciprocating actuator is automated by way of the processor.

In one exemplary embodiment of a method for shearing a nucleic acid, the method includes depositing a sample into a container, cycling the sample between the container, a cycle receiver, and an orifice located between the container and the cycle receiver to shear the sample, removing the sample from the container, and disposing of the container. Optionally, a second sample can then be deposited into a second container, cycled between the second container, a second cycle receiver, and an orifice located between the second container and the second cycle receiver to shear the sample, the sheared sample can be removed, and then the second container can be disposed. The method can also include setting a number of sample cycles, a flow rate, or both to control the approximate size of the sheared sample. In one embodiment the sheared sample is in the range of about 4 kilo-base pairs to about 40 kilo-base pairs. For example, the sheared sample can be greater than or equal to about 10 kilo-base pairs. In another embodiment a recovery rate of the sample can be greater than or equal to about 85 percent.

In one exemplary embodiment of a nucleic acid shearing kit, the kit includes a disposable syringe, an orifice structure, and a disposable, open-ended tubing. The disposable syringe can include a syringe body and a syringe plunger disposed therein, with the body defining a first reservoir to receive a nucleic acid sequence sample. The orifice structure can include a first end that is configured to be coupled to and in fluid communication with the syringe body. The orifice structure can also include a second end that is configured to be coupled to and in fluid communication with a second reservoir. The second reservoir can be defined by the disposable open-ended tubing and can be for receiving the sample following passage through the orifice structure. Further, the orifice structure can include at least one hole having a diameter in a range of about 25 micrometers to about 125 micrometers. The hole allows passage of the sample between the first reservoir and the second reservoir.

The kit can also include a number of other components. For example, the kit can include a first coupling component for coupling the syringe body to the first end of the orifice structure. The kit can also include a second coupling component for coupling the tubing to the second end of the orifice structure. In one embodiment the first coupling component includes a Luer adapter. In one embodiment the second coupling component includes a nut, a ferrule, and a lock ring.

A number of materials can be used. For example, in one embodiment the syringe body can include plastic. In another embodiment the tubing can include a polytetrafluoroehtylene (PTFE) tubing.

Still further, in some embodiments the kit can include more than one orifice structure and/or more than one disposable syringe. In instances in which there are multiple orifice structures, the orifice structures can have different sized diameters that produce different sizes of sheared samples when the sample is cycled through the orifice structure. In instances in which there are multiple disposable syringes, each syringe can be configured for use with a single sample.

### BRIEF DESCRIPTION OF DRAWINGS

This invention will be more fully understood from the following detailed description taken in conjunction with the accompanying drawings, in which:

FIG. 1A is a perspective view of one exemplary embodiment of a reservoir of a nucleic acid shearing cartridge;

FIG. 1B is a top view of the reservoir of FIG. 1A, including a needle for drawing a sample into the reservoir;

FIG. 2A is an exploded, perspective view of one exemplary embodiment of a nucleic acid shearing cartridge, including the reservoir of FIG. 1A;

FIG. 2B is an exploded, top view of the nucleic acid shearing cartridge of FIG. 2A

FIG. 3A is an assembled, perspective view of the nucleic acid shearing cartridge of FIG. 2A;

FIG. 3B is an assembled, top view of the nucleic acid shearing cartridge of FIG. 3A;

FIG. 4 is a perspective view of one exemplary embodiment of a nucleic acid shearing apparatus without any nucleic acid shearing cartridges disposed therein; and

FIG. 5 is a front view of the nucleic acid shearing apparatus of FIG. 4 having a plurality of nucleic acid shearing cartridges disposed therein.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Apparatuses, devices, and methods for shearing nucleic acid are generally provided. Nucleic acid samples such as DNA and RNA are typically sheared prior to sequencing them. The sample can be sheared by passing the sample through an orifice one or more times to achieve a desired sample length, which is typically measured in bases. In one exemplary embodiment, the sample to be sheared can be disposed in a first reservoir and passed through an orifice to a second reservoir. The sample can be cycled between the two reservoirs any number of times. Once the desired sample length is achieved, the sample can be removed from the reservoir and the reservoir discarded. The sample is now ready to be sequenced using any number of sequencing techniques. Another reservoir can be used as the starting location for a second sample to be sheared. In some instances the same orifice may be used to shear the second sample, while in other instances it may be desirable to use a new orifice, which can have the same size or a different size as the first orifice Likewise, the second reservoir can be reused, although in a preferred embodiment a new second reservoir is used to receive the sample from the new first reservoir.

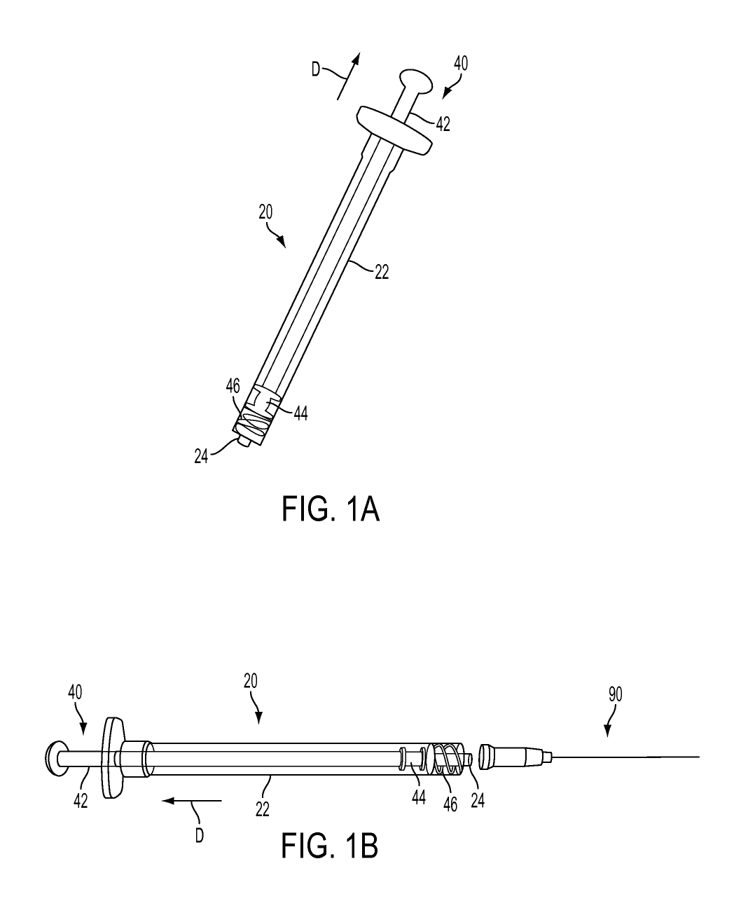

FIGS. 1A and 1B illustrate one embodiment of a first reservoir 20, alternatively referred to as a container, for use as part of a disposable nucleic acid shearing cartridge. FIG. 1A includes the reservoir 20 and a fluid driver 40, while FIG. 1B also includes a needle 90, which is one way by which a nucleic acid sample to be sheared can be introduced to the first reservoir 20. As illustrated, the first reservoir 20 is more commonly known as a syringe body 22 and the fluid driver 40, which is coupled to the syringe body 22, is more commonly known as a syringe plunger 42. Additional components common to syringes, such as a piston 44, a spring 46, and a coupling component 24, are also provided as part of the reservoir-fluid driver component. The coupling component 24 can be configured to hold liquid within the syringe body 22 when no force is acted upon it, but to permit liquid to flow through it when suction or an injection force is applied thereto. In the illustrated embodiment, the coupling component 24 is a male Luer lock.

As illustrated, the syringe body 22 is generally cylindrical and is capable of housing a sample of nucleic acid. The syringe body 22 can have a range of volumes, such as in the range of about 0.1 milliliters to about 3 milliliters, or more particularly in the range of about 0.5 milliliters to about 2 milliliters. In one embodiment the syringe body 22 defines a volume of at least about 0.5 milliliters. In another embodiment the syringe body 22 defines a volume of at least about 1 milliliter. While a variety of materials can be used to form the syringe body 22, materials that are inexpensive and easy to dispose of are preferred because the syringe body 22 is generally designed for a single use. This helps reduce the possibility of contamination, as well as increase accuracy and reproducibility. Any number of polymers can be used to help form the syringe body 22, however, in one exemplary embodiment the syringe body 22, as well as the coupling component 24, includes plastic.

The syringe plunger 42 can also have a variety of sizes and be made from a variety of materials. The size of the syringe plunger 42 will typically depend on the size of the syringe body 22, as the plunger 42 needs to be able to fit with the syringe body 22 so that suction can be created when the syringe plunger 42 is pulled and an injection force can be created when the syringe plunger 42 is pushed. Often the syringe plunger 42 is made from the same materials as the syringe body 22. Any number of polymers can be used, and in one embodiment the syringe plunger 42 is made of plastic. Other components, such as the piston 44 and the spring 46, can also have a variety of sizes and be made of a variety of materials. The size of these components is generally dependent on the size of the syringe body 22. In one exemplary embodiment the piston 44 is made of a rubber and the spring 46 is made of a metal. In one exemplary embodiment the combination of the syringe body 22, the syringe plunger 42, the coupling component 24, the piston 44, and the spring 46 is a 1 milliliter Luer Lock syringe from Becton, Dickinson and Company.

A sample to be sheared can be introduced into the syringe body 22 in any number of manners. FIG. 1B includes a needle 90, which in conjunction with the syringe plunger 42 and its associated components, such as the piston 44 and the spring 46, is one exemplary way by which a sample can be introduced into the syringe body 22. The needle 90 can be removably mated to the syringe body 22 by coupling to the coupling component 24. The needle 90 can then be placed in contact with the sample to be sheared and the syringe plunger 42 can be pulled in a direction D, away from the needle. This, in turn, causes suction, which draws the liquid into the syringe body 22. After the sample is loaded into the syringe body 22, the needle 90 can be disconnected from the coupling component 24 and removed. In one exemplary embodiment, the needle is a Type 304 stainless steel dispensing needle that is 26 gauge, has an inner diameter of about 254 micrometers, an outer diameter of about 457.2 micrometers, and a length of about 3.81 centimeters.

Although the first reservoir 20 and fluid driver 40 are illustrated as a syringe body and a syringe plunger, respectively, a person skilled in the art will recognize that a variety of other devices can be used to initially house the sample and introduce the sample into the first reservoir 20 for shearing without departing from the spirit of the invention. By way of non-limiting example, rather than a syringe body, the first reservoir could be a plastic tube and the fluid driver could be an eye dropper rather than a syringe plunger. Likewise, although the first reservoir 20 as described is generally cylindrical, has a range of preferred sizes, and is described as being made from a polymer such as plastic, any number of shapes, sizes, and materials can be used to form the first reservoir and the fluid driver.

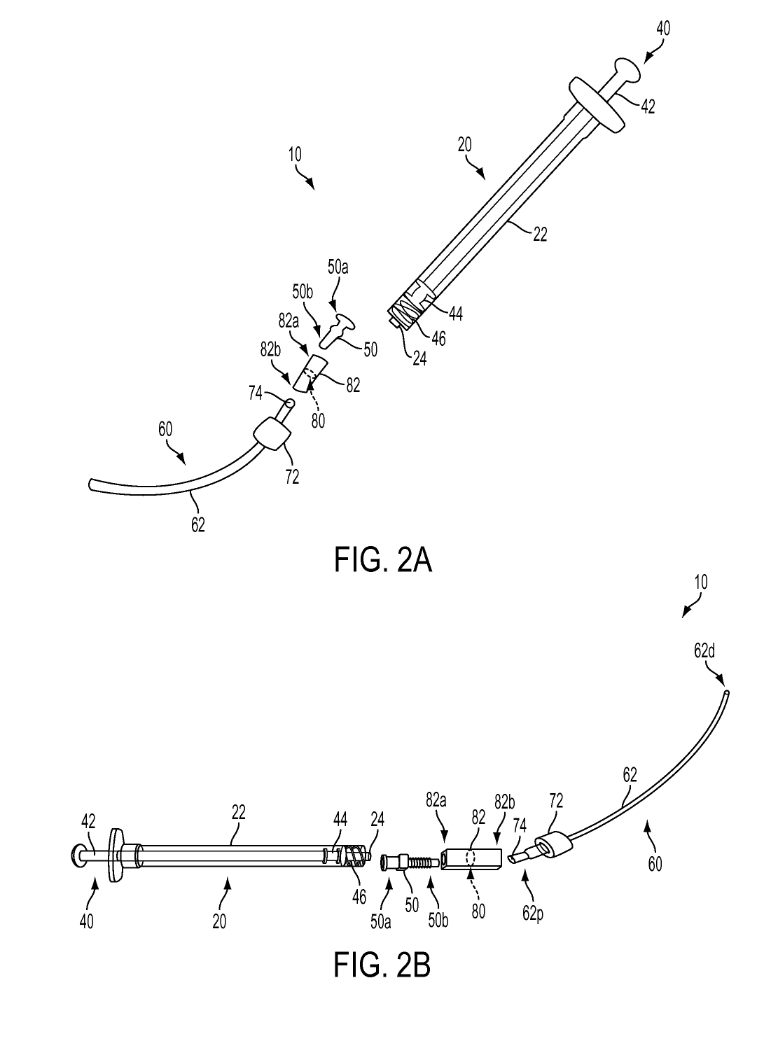

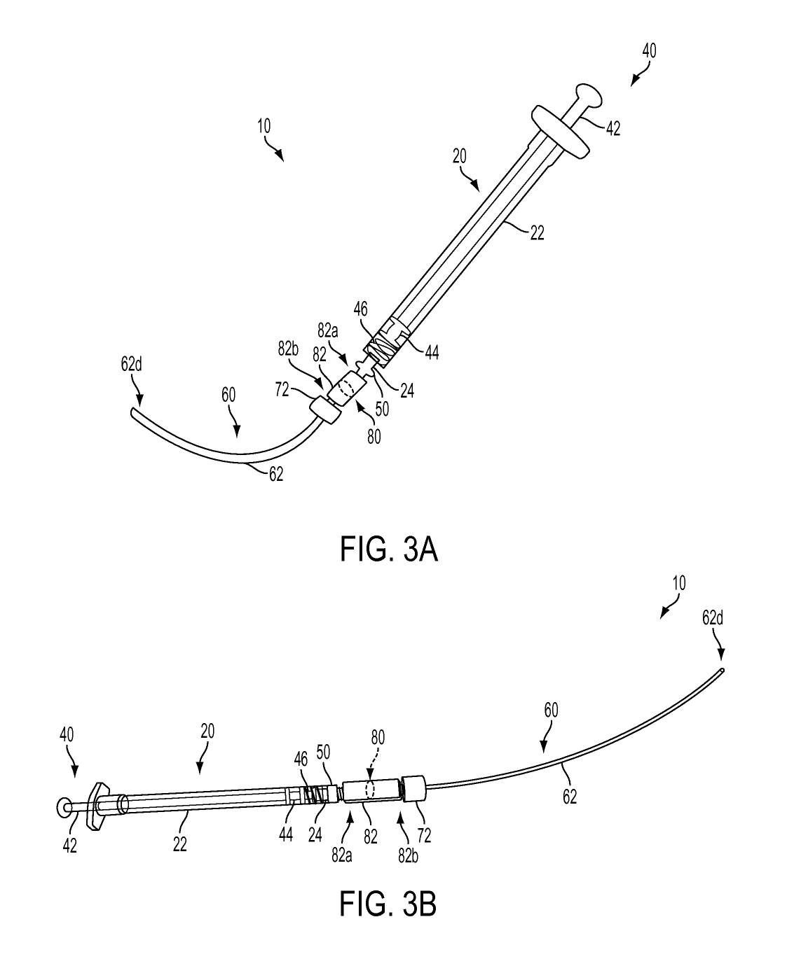

Once the sample is loaded into the first reservoir 20, additional components of a nucleic acid shearing cartridge 10 can be attached to the first reservoir 20, as shown in FIGS. 2A, 2B, 3A, and 3B. Alternatively, it is envisioned that one or more of these components could be coupled to the first reservoir 20 prior to introduction of the sample to the first reservoir 20, depending on the method used to introduce the sample into the reservoir 20. In addition to the first reservoir 20 and the fluid driver 40, the other two main components of the cartridge 10 are a second reservoir 60, alternatively referred to as a cycle receiver, and an orifice 80. The second reservoir 60 is configured to receive a sample that is ejected from the first reservoir 20 and through the orifice 80. The orifice 80 is contained within some sort of structural component, which in the illustrated embodiment is an orifice structure 82. In other embodiments the orifice may be part of the first or second reservoirs.

The second reservoir 60 can include any housing or container that is capable of receiving a fluid. In the illustrated embodiment, the second reservoir is a tubing 62 with open proximal and distal ends 62p, 62d. The open proximal end 62p of the tubing 62 can be coupled to the orifice structure 62 and the open distal end 62d of the tubing 62 can be left open in use. By allowing the distal end 62d to remain open, air can flow out of the system and not hinder the movement of fluid from the first reservoir 20 to the second reservoir 60.

The second reservoir 60 can be any number of sizes, but is typically large enough to receive an entire sample from the first reservoir 20 so that portions of the sample are not lost due to over-filling or unable to leave the first reservoir 20 or the orifice structure 82 because the second reservoir 60 is full. Like the other components of the cartridge 10, the size of one component generally depends on the size of the other components, which in turn can depend on the size of the sample to be sheared and the desired size of the sheared sample. In one exemplary embodiment the tubing 62 has an inner diameter that is approximately 1.5748 millimeters, an outer diameter that is about 3.175 millimeters, and a length that is about 15 centimeters. Further, similar to the first reservoir 20, the second reservoir 60 can also be disposable so that the second reservoir 60 is only used once. This helps reduce the possibility of contamination, as well as increase accuracy and reproducibility. Any number of polymers can be used to help form the second reservoir 60, however, in one exemplary embodiment the tubing 62 is made of a high purity perfluroalkoxy (PFA).

In the illustrated embodiment, the orifice 80 is contained within the orifice structure 82. A number of shapes and sizes of the orifice 80 and the orifice structure 82 can be used. The shape and size of the orifice 80 can affect the size of the resulting sheared sample, alternatively referred to as the resulting sheared fragment. For example, in one embodiment, an orifice can be substantially circular and have a diameter that is about 50.8 micrometers can yield a sheared sample of about 3 kilo-base pairs, while a substantially circular orifice having a diameter that is about 88.9 micrometers can yield a sheared sample of about 8 kilo-base pairs. In other embodiments the orifice can have a different shape and/or a different size, for example, the orifice can be substantially triangular or rectangular. Likewise, because other factors can also effect the size of the resulting fragment, such as the size of the initial sample, the flow rate of the sample through the orifice 80, and the number of times the sample is cycled through the orifice 80, one skilled in the art will recognize that two orifices having the same size can yield different sized resulting fragments.

The orifice structure 82 containing the orifice 80 can generally be sized to form a fluid tight seal between the first and second reservoirs 20, 60. In the illustrated embodiment the orifice structure 82 is generally in the shape of a rectangular prism, although any other number of shapes can be used. Further, the orifice structure 82 can be made from a variety of materials. Inorganic materials can be particularly useful in this context. Some examples of inorganic materials that can be used to form the orifice structure 82, and the orifice 80 contained therein, include glasses, ceramics, and any number of crystalline materials. In one exemplary embodiment the orifice structure 82 includes sapphire. In the illustrated embodiment the orifice structure 82 is a Bird Precision Orifice having an orifice with a diameter that is about 50.8 micrometers.

A first end 82a of the orifice structure 82 can be configured to mate to the first reservoir 20 and a second end 82b of the orifice structure 80 can be configured to mate to the second reservoir 60. Mating between these three components can occur in a variety of matters. For example, in some embodiments the orifice structure 82 can mate directly to one or both of the first and second reservoirs 20, 60. In other embodiments, such as the embodiment illustrated in FIGS. 2A and 2B, one or more coupling components can be used to facilitate a fluid-tight seal between the orifice structure 82 and the first and second reservoirs 20, 60. As shown, a first coupling component is a Luer adapter 50 having a female Luer to M6 male Luer lock. A first end 50a of this Luer lock is configured to mate with the coupling component 24 of the syringe body 22 to form a fluid-tight seal, and a second end 50b of this Luer lock is configured to mate with the first end 82a of the orifice structure 82. Further, as shown, a second coupling component includes a nut 72, a ferrule 74, and a lock ring (not shown). In the illustrated embodiment the nut 72 is a M6 fitting Click-N-Seal adapter having a diameter that is about 3.175 millimeters, the ferrule 74 is a polyetheretherketone (PEEK) ferrule, and the lock ring is made of stainless steel. These three components 72, 74, and 76 can operate together to form a fluid-tight seal between the orifice structure 82 and the second reservoir 60, for instance by crimping the lock ring and ferrule 74 to an end of the tubing 62 and sliding the nut 72 on the tubing 62, over the location of the lock ring and ferrule 74. A person having skill in the art will recognize that any number of adapters and fittings can be used to form coupling components between the orifice structure 82 and the first and second reservoirs 20, 60 Likewise, in some embodiments the coupling components can be part of the reservoirs 20, 60 or the orifice structure 82 themselves.

FIGS. 3A and 3B illustrate the cartridge 10 when it is fully assembled, however without any fluid disposed in the first reservoir 20. In the illustrated embodiment, each of the components of the cartridge 10 is made from inexpensive and readily available components and materials so that the cartridge 10 is only used for a single sample. This results in the reduction of the possibility of contaminating the sample, and also improves repeatability and accuracy of obtaining the desired sheared sample size. Nevertheless, the components of the disclosed cartridge 10 can be reused if desired. For example, it may be desirable to reuse the orifice structure 82 a number of times, as doing so may prove to result in consistent shearing between samples because the same orifice 80 is used.

Although the disclosed cartridge 10 can be operated manually, for instance by pumping the syringe plunger 42 back and forth to cycle the sample from the first reservoir 20, through the orifice 80, to the second reservoir 60, and then back through the orifice 80 and to the first reservoir 20, the present invention also includes a nucleic acid shearing apparatus that can perform the cycling mechanically, without physical forces being supplied by an operator. In one exemplary embodiment, the apparatus is both automated and capable of processing multiple samples in parallel.

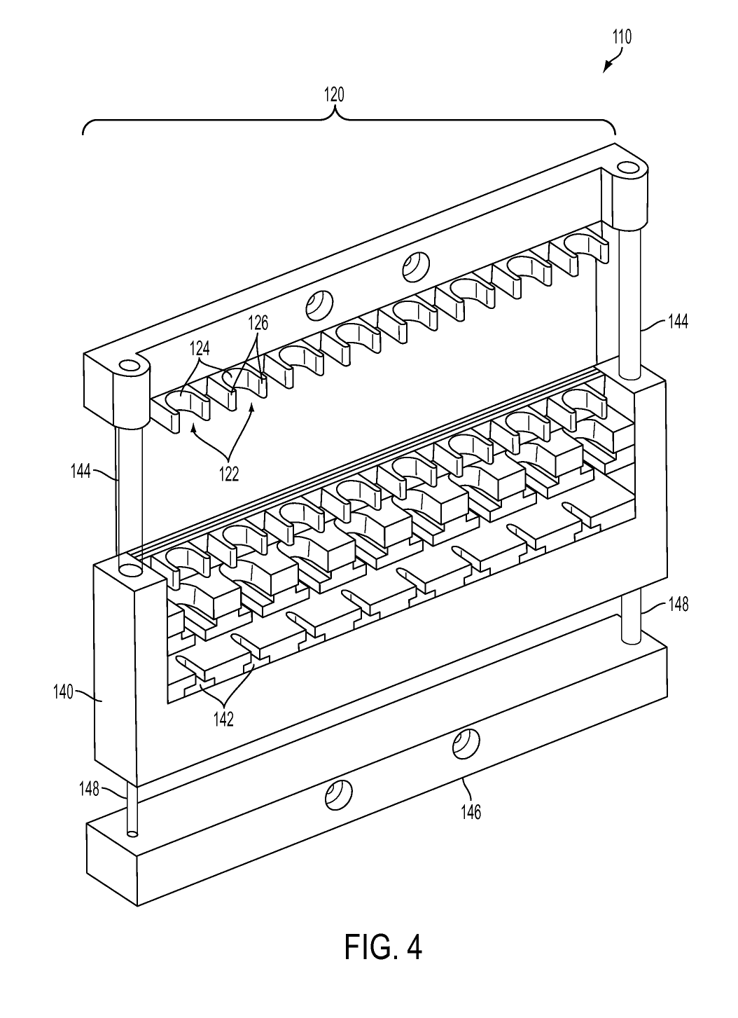

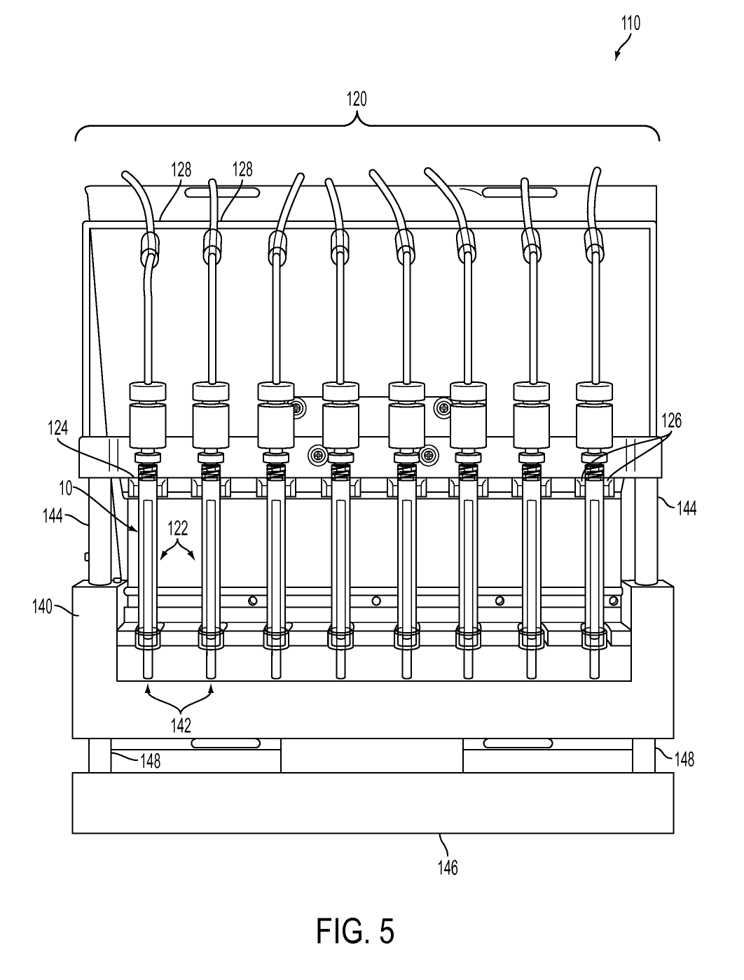

As shown in FIGS. 4 and 5, the apparatus 110 can include a housing 120 for receiving a nucleic acid shearing cartridge and a reciprocating actuator 140 for engaging a fluid driver of the cartridge to cause the back-and-forth cycling between reservoirs of the cartridge. The apparatus 110 can also include a processor (not illustrated) that can help control the various factors that affect the resulting size of the sheared sample. The processor can be linked to the reciprocating actuator 140 to start and stop the actuator 140 based on the input provided to the processor.

The housing 120 of the illustrated embodiment actually includes eight chambers 122 for receiving a cartridge. As illustrated, the chambers 122 can be configured to hold cartridges like the cartridges 10 illustrated in FIGS. 1A-3B, although certainly the chambers 122 can be adapted to hold a variety of types of cartridges. The illustrated housing 120 includes first reservoir holders 124, each having two cartridge-engaging arms 126 configured to hold a portion of the first reservoir of the cartridge in place. Optionally, second reservoir holders 128 can be included, as shown in FIG. 5, to maintain a relative position of a second reservoir of the cartridges. In the illustrated embodiment the second reservoir holders 128 are apertures. In other embodiments the housing 120 may only include a single chamber, two chambers, more than two chambers, and even more than eight chambers. By including multiple chambers, multiple samples can be sheared simultaneously, i.e., in parallel. Not only does this vastly increase the throughput in comparison to current shearing techniques, but it improves accuracy and consistency at least because when samples are run at different times, there is a greater possibility that the force applied by the reciprocating actuator 140 is not consistent in each run.

The reciprocating actuator 140 is configured to engage a fluid driver of a cartridge to cause a sample within a first reservoir of the cartridge to pass through an orifice of the cartridge and into a second reservoir of the cartridge, and then to return the sample back through the orifice and to the first reservoir. This operation can occur a multitude of times. As illustrated, the reciprocating actuator 140 includes a plurality of fluid driver receivers 142 that allow the actuator 140 to induce both a pushing action to eject the fluid from a first reservoir of a cartridge and a pulling action to suction fluid back into the first reservoir. Pistons 144 can be disposed between the housing 120 and the reciprocating actuator 140 to assist in moving the reciprocating actuator 140. A base 146 can optionally be included. The base 146 can create a space between the ground and the reciprocating actuator 140 so that the reciprocating actuator 140 does not contact the ground when it is in a position closest to the ground. In the illustrated embodiment the base 146 is coupled to the reciprocating actuator by stands 148. In one exemplary embodiment, the apparatus 110 includes components of a Kloehn V6 Multi-Channel Syringe Pump.

The processor can be capable of modifying a number of the different variables that affect the size of the resulting sheared sample. Some of these factors include a size of the original sample, a size of the orifice, the flow rate of the fluid through the orifice, and the number of times the sample is cycled through the orifice. Other options can also be included as part of the processor, such as a manual aspirate mode to load the sample into the reservoir and an option to dispense the sample after the cycles are complete. Parameters inputted into the processor to control the operation of the apparatus 110 can be communicated to the illustrated apparatus 110 by way of a serial connection. In the illustrated embodiment the serial connection is a RS-232 serial connection, although any number of connection protocols can be used, such as a USB connection.

For example, the effects of one of the identified factors is that as the size of an orifice increases, so too does the size of the resulting fragment. Thus, in one embodiment, when the diameter of an orifice is about 40.64 micrometers, the resulting fragment is about 2 kilo-base pairs, when the diameter of the orifice is about 50.8 micrometers, the resulting fragment is about 3 kilo-base pairs, when the diameter of the orifice is about 63.5 micrometers, the resulting fragment is about 4 kilo-base pairs, when the diameter of the orifice is about 76.2 micrometers, the resulting fragment is about 6 kilo-base pairs, and when the diameter of the orifice is about 88.9 micrometers, the resulting fragment is about 8 kilo-base pairs.

By way of further example, as the flow rate of the sample through the orifice decreases, the size of the resulting fragment increases. Thus, in one embodiment, when the flow rate of the sample is about 100 microliters per second, the resulting fragment is about 2 kilo-base pairs, when the flow rate of the sample is about 80 microliters per second, the resulting fragment is about 2.9 kilo-base pairs, when the flow rate of the sample is about 50 microliters per second, the resulting fragment is about 3.5 kilo-base pairs, when the flow rate of the sample is about 40 microliters per second, the resulting fragment is about 4.2 kilo-base pairs, and when the flow rate of the sample is about 30 microliters per second, the resulting fragment is about 5.2 kilo-base pairs.

By way of one last example, as the number of cycles increases, the size of the resulting fragment decreases. A cycle for this example is considered movement from either the first reservoir to the second reservoir or from the second reservoir to the first reservoir, and thus movement of the sample from the first reservoir, to the second reservoir, and back to the first reservoir is considered two cycles. Thus, in one embodiment, when 6 cycles are completed, the resulting fragment is about 4.2 kilo-base pairs, when 12 cycles are completed, the resulting fragment is about 3.9 kilo-base pairs, when 18 cycles, 22 cycles, or 24 cycles are completed, the resulting fragment is about 2.8 kilo-base pairs. Thus, typically there is a certain number of cycles after which no significant shearing occurs. In the provided example, after about 18 cycles, no significant shearing was detected. A person skilled in the art will appreciate that how many cycles should be run before no significant shearing occurs depends on the many other factors that affect the resulting fragment size.

Although some of the examples provided demonstrate the ability to yield sheared sample sizes between about 2 kilo-base pairs and about 8 kilo-base pairs, these factors can be optimized to yield kilo-base pairs in the range of about 2 kilo-base pairs to about 40 kilo-base pairs, and at least greater than or equal to 10 kilo-base pairs.

In use, the cartridge 10 is filled with a desired sample size and its components are coupled together so that the cartridge 10 includes two reservoirs 20, 60 and an orifice 80 disposed therebetween. The cartridge 10 is then loaded into a chamber 122 of the apparatus 110 for shearing nucleic acid by engaging the first reservoir 20 with the cartridge-engaging arms 126 of the first reservoir holder 124. This procedure can be performed for however many samples are going to be run in parallel. Once all of the cartridges 10 are loaded into the apparatus 110, a user can input parameters to the processor to obtain a desired sheared fragment. For example, in one embodiment the operator can input the volume of the initial sample, the number of cycles to be performed, and a speed rating that is indicative of the rate at which the sample will flow through orifice. In one experiment that was conducted, the volume of the initial sample was 5 micrograms mouse genomic DNA in 200 microliters and the diameter of the orifice was approximately 50.8 micrometers. 22 cycles were conducted at a speed rating of 7, which translated to a flow rate of about 45.8 microliters per second. Once the cycles were completed, the cartridge 10 was removed from the apparatus 110 and the sample extracted from the first reservoir 20. The resulting fragment was about 10 kilo-base pairs. Further, the recovery rate was greater than about 85 percent. The recovery rate is the amount of sample that came out of the system. In systems of this nature, some of the sample is lost due to the sample being left behind on other components of the cartridge 10, however, in earlier versions of shearing devices, the recovery rate typically did not exceed 80 percent. After the sample is extracted from the first reservoir 20, the first reservoir 20 can be disposed of and new cartridges 10 can be filled with samples and used in conjunction with the apparatus 110. These second samples can be processed in a similar manner and then extracted for use, for example to be sequenced. The first reservoirs 20 used in conjunction with the second samples can also be disposed of after the sample is recovered.

The present invention also results in a kit for shearing nucleic acid. The kit can include the cartridge itself, and thus can include a disposable syringe that serves as the first reservoir and the fluid driver, an orifice structure having an orifice for shearing nucleic acid, and a disposable, open-ended tubing that serves as the second reservoir. The components of the kit can have properties similar to those discussed above with respect to the cartridge 10 illustrated in FIGS. 1A-3B. For example, the orifice structure can include at least one hole having a diameter in a range of about 25 micrometers to about 125 micrometers, or more particularly in the range of about 40 micrometers to about 100 micrometers. The kit can also include one or more coupling components. In one embodiment, a first coupling component can be provided for coupling the syringe body to the first end of the orifice structure and a second coupling component can be provided for coupling the tubing to the second end of the orifice structure. Coupling components such as the components discussed above with respect to the cartridge 10 can be provided, such as a Luer adapter for the first coupling component and a nut, a ferrule, and a lock ring for the second coupling component.

In some embodiments, the kit may include multiple orifice structures. The orifice structures can have different sized orifices, thereby allowing an operator to choose the orifice structure best-suited for achieving the desired fragment size. In fact, any component of the kit can be provided as multiple components. For instance, the kit can include multiple disposable syringes, so that the syringes can be disposed of after a single use.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A disposable nucleic acid shearing cartridge, comprising:
a first reservoir for receiving a nucleic acid sequence sample;
an orifice structure in fluid communication with the first reservoir;
a second reservoir also in fluid communication with the orifice structure for receiving the sample following passage through the orifice structure; and
a fluid driver for cycling the sample between the first and second reservoirs a plurality of times to shear the nucleic acid into pieces of desired lengths.

2. The disposable cartridge of claim 1, wherein the first reservoir is a syringe body and the fluid driver is a syringe plunger coupled to the syringe body.

3. The disposable cartridge of claim 1, wherein the first reservoir defines a volume of at least about 0.5 milliliters.

4. The disposable cartridge of claim 1, wherein the first reservoir defines a volume of at least about 1 milliliter.

5. The disposable cartridge of claim 1, wherein the orifice structure further comprises an inorganic material having at least one fluid-passing hole therethrough.

6. The disposable cartridge of claim 5, wherein the hole has a diameter in a range of about 25 micrometers to about 125 micrometers.

7. The disposable cartridge of claim 5, wherein the hole has a diameter in a range of about 50 micrometers to about 100 micrometers.

8. The disposable cartridge of claim 5, wherein the inorganic material of the orifice structure comprises a material selected from glasses, ceramics, and crystalline materials.

9. The disposable cartridge of claim 8, wherein the inorganic material of the orifice structure comprises sapphire.

10. The disposable cartridge of claim 1, wherein at least one of the first and second reservoirs comprises a plastic body.

11. A nucleic acid shearing apparatus, comprising:
a housing for receiving at least one nucleic acid shearing cartridge, the cartridge comprising a first reservoir for receiving a nucleic acid sequence sample, an orifice structure in fluid communication with the first reservoir, a second reservoir also in fluid communication with the orifice structure for receiving the sample following passage through the orifice structure, and a fluid driver for cycling the sample between the first and second reservoirs;
a reciprocating actuator configured to engage the fluid driver of the cartridge to cause a sample within the first reservoir to pass through the orifice structure into the second reservoir and then to return to the first reservoir; and
a processor for controlling at least one of a flow rate of the sample and a number of times the sample passes through the orifice structure.

12. The apparatus of claim 11, wherein the housing comprises a plurality of receptacles for receiving a plurality of cartridges and processing multiple samples in parallel.

13. The apparatus of claim 11, wherein the reciprocating actuator is automated by way of the processor.

14. A method for shearing a nucleic acid, comprising:
depositing a sample into a container;
cycling the sample between the container, a cycle receiver, and an orifice located between each of the container and the cycle receiver to shear the sample;
removing the sheared sample from the container; and
disposing of container.

15. The method of claim 14, further comprising:
depositing a second sample into a second container;
cycling the second sample between the second container, a second cycle receiver, and an orifice located between each of the second container and the second cycle receiver to shear the sample;
removing the sheared sample from the second container; and
disposing of the second container.

16. The method of claim 14, further comprising:
setting at least one of a number of sample cycles and a flow rate to control the approximate size of the sheared sample.

17. The method of claim 14, wherein the sheared sample is in the range of about 4 kilobases to about 40 kilobases.

18. The method of claim 14, wherein the sheared sample is greater than or equal to about 10 kilobases.

19. The method of claim 14, wherein a recovery rate of the sample is greater than or equal to about 85 percent.

20. A nucleic acid shearing kit, comprising:
a disposable syringe comprising a syringe body and syringe plunger disposed therein, the body defining a first reservoir to receive a nucleic acid sequence sample;
an orifice structure comprising a first end configured to be coupled to and in fluid communication with the syringe body, a second end configured to be coupled to and in fluid communication with a second reservoir, and at least one hole having a diameter in a range of about 25 micrometers to about 125 micrometers; and
a disposable, open-ended tubing defining a second reservoir for receiving the sample following passage through the orifice structure.

21. The kit of claim 20, further comprising:
a first coupling component for coupling the syringe body to the first end of the orifice structure; and
a second coupling component for coupling the tubing to the second end of the orifice structure.

22. The kit of claim 21, wherein the first coupling component comprises a Luer adapter.

23. The kit of claim 21, wherein the second coupling component comprises a nut, a ferrule, and a lock ring.

24. The kit of claim 20, wherein the syringe body comprises plastic.

25. The kit of claim 20, wherein the tubing comprises a polytetrafluoroehtylene (PTFE) tubing.

26. The kit of claim 20, wherein the orifice structure comprises a plurality of orifice structures having different sized diameters that produce different sizes of sheared samples when the sample is cycled therethrough.

27. The kit of claim 20, wherein the disposable syringe comprises a plurality of disposable syringes, each syringe being configured for use with a single sample.
